(19) United States
(12) Patent Application Publication
(10) Pub. No.: US 2007/0195926 A1
(43) Pub. Date: Aug. 23, 2007

(54) CT method for recording projection data

(76) Inventors: Martin Munker, Gevelsberg (DE); Oliver Rokitta, Ennepetal (DE); Matthias Helling, Heme (DE)

Correspondence Address: GRAYBEAL, JACKSON, HALEY LLP, 155 - 108TH AVENUE NE, SUITE 350, BELLEVUE, WA, 98004-5901, US

(21) Appl. No.: 11/603,620

(22) Filed: Nov. 21, 2006

(30) Foreign Application Priority Data
Nov. 21, 2005 (DE) 10 2005 055 423.7

(51) Int. Cl. H05G 1/60; A61B 6/00; G01N 23/00; G21K 1/12

(52) U.S. Cl. 378/19

(57) ABSTRACT

The invention relates to a CT method for recording projection data of an object for examination in the field of non-medical applications by means of X-rays in which projection data for different angular positions of the object for examination are required. It is provided according to the invention that the integration time for recording each angular position depends on the geometry of the object for examination and the adsorption properties of the object for examination in this angular position.

Priority to German patent application number DE 10 2005 055 423.7 filed on 21 Nov. 2005 under 35 U.S.C. §119(a) is claimed, said application being incorporated herein by reference.

### FIELD OF THE INVENTION

The invention relates to a CT method for recording projection data of an object for examination in the field of non-medical applications by means of X-ray beams, in which projection data for different angular positions of the object for examination are acquired.

### BACKGROUND OF THE INVENTION

With the known CT measurements the object is rotated in an X-ray fan or cone of X-rays. Rotation takes place either stepwise, wherein the same period of time is always spent in defined angular positions, or else the object is rotated continuously and, with the exception of the acceleration ramps, evenly. With the continuous method the object is thus rotated at equal time intervals about identical angles. With the stepwise method the integration time is identical in each angular position.

With the continuous method free process dimensionings are common with the result that the actual position is not synchronized with the read-out of the detector and fluctuations in the rotational speed (flutters) attributable to equipment are neither recorded nor corrected.

The rotational speed and the integration time must be set such that the maximum achievable resolution and the minimum signal-to-noise ratio is achieved for each angular position—i.e. also for the most unfavourable positions. An extremely high measurement time is thereby obtained, as the rotational speed and the integration time must be geared to the worst case.

### SUMMARY OF THE INVENTION

An object of the invention is to provide a CT method with which, where heterogeneous objects are to be examined, a reduction of the measurement time can be achieved while quality remains constant.

This object is achieved by a CT method with the features of claim 1. Because the integration time for recording each angular position depends on the geometry of the object for examination and the absorption properties of this object for examination in this angular position, the overall measurement time for the object for examination is optimized. It is thereby possible, in an angular position in which there is only a very low absorption, to pass quickly to the next angular position with a short integration time. On the other hand it is possible, in an angular position in which a high absorption through the object for examination occurs, to measure for a long time with the result that a sufficiently good measurement result is obtained. This prevents the occurrence of artifacts or the spending of an unnecessarily long time in a specific angular position.

An advantageous development of the invention provides that there are constant angular increments between the angular positions. The size of the angular increments (or the number of projections) depends on the number of detector elements (Nyquist-Shannon sampling theorem).

A further advantageous development of the invention provides that projection data are recorded at a constant sampling rate. The acquired data can be more simply evaluated by using a constant sampling rate. Instead of matching the sampling rate to the absorption conditions of the studied item for examination—these are in particular the penetrability and the signal-to-noise ratio—the residence time at a defined projection angle in stop & go mode or a modulated time for sweeping an angular range with continuous rotation is preferred.

Particularly preferably when an image-recording system has a fixed time setting several results are totalled which lie in a predeterminable angular range about the associated angular position. The movement need not thereby be stopped at a discrete angular position, but several measurements can be assigned to one angular position in a continuous process with the result that the signal-to-noise ratio is increased for this angular position.

A further advantageous development of the invention provides that the integration time varies with the rotational speed of a rotation unit. A sufficient signal-to-noise ratio or a minimum counting rate can thereby always be achieved.

A further advantageous development of the invention provides that during the measurement the voltage and/or the current of the X-ray source are kept constant. The effect of the constant voltage is that the spectral distribution of the emitted X-radiation does not change and thus no complicated correction calculations need be undertaken. The maintaining of a constant current strength guarantees that the scatter ratios and imaging properties of the whole system are not changed.

Advantageously, measurement continues in each angular position until a predeterminable minimum signal-to-noise ratio is reached. It is thereby ensured that the image quality in this angular position is good enough and no artifacts occur at this point. Because this takes place for every angular position, with a simultaneously minimal overall measurement time the signal-to-noise ratio as required for image quality is sufficient at any point.

Furthermore it is advantageous if the same signal-to-noise ratio is reached in each angular position. The image quality is thereby the same throughout all angular positions and thus a minimum overall examination time of the object is achieved for the set image quality.

Another advantageous development of the invention provides that in every angular position a signal value is achieved which lies between a predeterminable minimum value and a predeterminable maximum value. The effect of this optimization criterion is that in all cases an offset which is inherent in the equipment is overcome and one remains below a signal value which would bring the detector to the limit of its recording capacity or even beyond it.

A further advantageous development of the invention provides that a synchronization takes place between a rotation unit and an image-recording system of the CT device throughout the recording. It is thereby guaranteed that in particular with a multi-stage measurement (see below) or with serial testing (see below) an exact allocation of the integration to the respective angular position is always possible even if the CT device is subjected to flutter. The method according to the invention can be used with any CT device.

A further advantageous development of the invention provides that, for serial testing of a known object, the integration times for the individual angular positions are determined in advance by means of a reference part and the rotational speed is then varied accordingly or the residence time is adjusted depending on the angular position. The optimum conditions for the respective angular position are ascertained using one or more reference parts and stored as a sub-routine of the examination program for the course of the scan. The rotational speed is then modulated accordingly in the case of continuous rotation. The residence time is varied for each examination position in the case of a measurement in defined angular positions, also called stop&go mode. Here, the minimum signal-to-noise ratio in all projections, already given above, or a signal-to-noise ratio that is the same everywhere is preferred as optimization criterion.

A further advantageous development of the invention provides that, when examining an unknown object, a single-stage measurement is carried out in which the projections are recorded in quick succession and, depending on absorption data obtained in an in-line evaluation, the rotational speed is varied accordingly or the residence time in the respective angular position is adapted. With this method the projections are incorporated in the data record true to position. A drag error, present in spite of the small delay, of the setting, is tolerated as it is only very small. The single-stage measurement is suitable in particular for objects for examination with moderate absorption changes.

A further advantageous development of the invention provides that when examining an unknown object, a multi-stage measurement is carried out, by performing a rapid base line measurement over all angular positions and then carrying out further projections at points at the angular positions where the image quality is not yet adequate. Unlike with the previously-described single-stage measurement there are no drag errors here as, after the first rapid measurement, the variations in rotational speed or the respective residence times in the defined angular positions can be calculated in advance and then adjusted or applied accordingly. An optimized image quality is thereby achieved even when there are pronounced absorption jumps. This means that in angular positions with high absorptions a very long residence time or an extremely low rotational speed can be used quite selectively, whereas in angular positions with low absorption the residence time can be kept very short or this angular position passed through at a high rotational speed.

For both the single-stage and the multi-stage measurement method, the reaching of a minimum signal value (which lies above the offset) which is to be freely defined and/or a maximum signal value (which lies below the value which the detector can no longer support) or a minimum signal-to-noise ratio, are used as optimization criteria in all projections.

With all methods the measured data can optionally be classified position-related (fixed angular increments, different integration times) or time-related (fixed integration time, varying angular increments) and are corrected accordingly for the following or concurrent reconstruction. With all methods, the overall measurement time is defined as the integrated time which the object spends either in each individual angular position (plus the idle times, for example for positioning and detector release) or needs for a complete revolution depending on the different angular velocities. Thereby a significantly quicker examination is achieved compared with the conventional, known methods as, in the case of a stepwise measurement according to the known methods, the overall measurement time is the product of the number of projections (the sampling rate) multiplied by the constant integration time per projection (then there are of course also the idle times). The sample rate here defines the maximum achievable resolution and cannot be chosen too small, as otherwise artifacts occur. The signal-to-noise ratio and thus the useful signal are directly influenced via the integration time. As the integration time must be set to the worst case, i.e. the angular position at which the highest absorption occurs, “much too long” is spent measuring in the positions in which only a small absorption occurs. Although a very high signal-to-noise ratio and also a large useful signal are thereby obtained, this is not necessary in order to properly examine the object for examination.

The time saved by the method according to the invention with sufficiently good examination quality is striking, in particular in the case of objects for examination which have pronounced absorption jumps. But even in the case of objects for examination with only moderate absorption changes, overall examination times are still achieved which lie clearly below those which are achieved with the known CT methods.

What is claimed:

1. CT method for recording projection data of an object for examination in the field of non-medical applications by means of X-rays in which projection data for different angular positions of the object for examination are acquired,
wherein,
the integration time for recording each angular position depends on the geometry of the object for examination and the absorption properties of the object for examination in this angular position.

2. Method according to claim 1, wherein there are constant angular increments between the angular positions.

3. Method according to claim 1, wherein projection data are recorded at a constant sampling rate.

4. Method according to claim 1, wherein, when an image-recording system has a fixed time setting several sub-results are totalled which lie in a predeterminable angular range about the associated angular position and are allocated to this.

5. Method according to claim 1, wherein the integration time is varied with the rotational speed of a rotation unit.

6. Method according to claim 1, wherein during the measurement the voltage and/or the current of the X-ray source are kept constant.

7. Method according to one claim 1, wherein a predeterminable signal-to-noise ratio is reached in each angular position.

8. Method according to claim 1, wherein the same signal-to-noise ratio is reached in each angular position.

9. Method according to claim 1, wherein in each angular position a signal value is reached which lies between a predeterminable minimum value and a predeterminable maximum value.

10. Method according to claim 1, wherein a synchronization takes place between the rotation unit and an image-recording system of the CT device throughout the recording.

11. Method according to claim 1, wherein, upon serial testing of a known object, the integration times for the individual angular positions are determined in advance by means of a reference part and the rotational speed is then varied accordingly or the residence time is adjusted depending on the angular position.

12. Method according to claim 1, wherein, when examining an unknown object, a single-stage measurement is carried out in which the projections are recorded in quick succession and, depending on absorption data obtained in an in-line evaluation, the rotational speed is varied accordingly or the residence time in the respective angular position is adjusted.

13. Method according to claim 1, wherein, when examining an unknown object, a multi-stage measurement is carried out by conducting a rapid base line measurement over all angular positions and then carrying out further projections at points at the angular positions where the image quality is not yet adequate.

14. Method according to claim 1, wherein instead of rotating the object for examination in a stationary CT device, the CT device is rotated about a stationary object.
